(19) United States
(12) Patent Application Publication
(10) Pub. No.: US 2025/0172449 A1
(43) Pub. Date: May 29, 2025

(54) PHYSIOLOGICAL INFORMATION MEASUREMENT SYSTEM AND PHYSIOLOGICAL INFORMATION MEASUREMENT METHOD

(71) Applicant: Nihon Kohden Corporation, Tokyo (JP)

(72) Inventors: Mariko ITO, Tokorozawa-shi (JP); Hirohito IGARASHI, Tokorozawa-shi (JP); Fumiyuki MATSUMURA, Tokorozawa-shi (JP); Minori HOSOI, Tokorozawa-shi (JP)

(73) Assignee: Nihon Kohden Corporation, Tokyo (JP)

(21) Appl. No.: 18/947,676

(22) Filed: Nov. 14, 2024

(30) Foreign Application Priority Data
Nov. 24, 2023 (JP) 2023-199033

(51) Int. Cl. G01L 27/00; G01L 23/32; G08B 21/18

(52) U.S. Cl. CPC G01L 27/007; G01L 23/32; G08B 21/182

(57) ABSTRACT

A physiological information measurement system includes: an obtaining unit configured to obtain, from a plurality of sensors that are connected to an air bag containing air and measure pressures received by the air bag from a subject, signals related to the measured pressures; and a determination unit configured to determine at least one of deterioration and failure of at least one of the sensors based on the signals related to the pressures obtained from the plurality of sensors.

### CROSS-REFERENCE TO RELATED APPLICATIONS

This application is based on and claims priority under 35 USC 119 from Japanese Patent Application No. 2023-199033 filed on Nov. 24, 2023, the contents of which are incorporated herein by reference.

### TECHNICAL FIELD

The presently disclosed subject matter relates to a physiological information measurement system, a physiological information measurement method, and a physiological information measurement program.

### BACKGROUND ART

In a medical facility such as a hospital, it may be necessary to noninvasively measure physiological information such as a heart beat or respiration of a patient lying on his/her back or lying his/her side on a bed without moving the patient. In a nursing home or the like, it is necessary to prevent a user from walking around and ensure safety of the user by a limited number of staff. To cope with such situations, there is known a technique (for example, JP2004-159804A) in the related art, in which an air bag is spread under a mattress laid on a bed, a pressure applied to the air bag from a patient who lies on his/her back or lying his/her side is detected by a pressure sensor, a microphone, or the like, and a heart beat, respiration, or the like of the patient on the bed is measured. In this technique, whether the patient gets in or out of bed is determined by comparing an amplitude of physiological information such as the detected respiration or heart beat with a predetermined threshold. In addition, a product has been developed in recent years, which analyzes states of a patient on a bed such as sleeping and awake, getting in bed and out of bed, in bed and out of bed, and changes in respiration and a heart beat from a measured body motion waveform, and notifies a medical worker of changes in conditions of the patient.

However, in the related art, since it is not possible to determine deterioration or failure of the pressure sensor, it is not possible to classify whether a change in the pressure waveform measured by the pressure sensor is caused by the deterioration or the failure of the pressure sensor or by the change in condition of the patient. Therefore, there is a problem that the condition of the patient on the bed cannot be accurately grasped.

### SUMMARY

The presently disclosed subject matter has been made to solve such a problem. That is, an object of the presently disclosed subject matter is to provide a physiological information measurement system, a physiological information measurement method, and a physiological information measurement program capable of determining whether a change in a measured pressure waveform is caused by deterioration or failure of a sensor or by a change in condition of a measurement subject.

The above problems of the presently disclosed subject matter are solved by the following methods.
(1) A physiological information measurement system includes: an obtaining unit configured to obtain, from a plurality of sensors that are connected to an air bag containing air and measure pressures received by the air bag from a subject, signals related to the measured pressures; and a determination unit configured to determine at least one of deterioration and failure of at least one of the sensors based on the signals related to the pressures obtained from the plurality of sensors.
(2) A physiological information measurement method includes: obtaining, from a plurality of sensors that are connected to an air bag containing air and measure pressures received by the air bag from a subject, signals related to the measured pressures; and determining at least one of deterioration and failure of at least one of the sensors based on the signals related to the plurality of pressures obtained from the plurality of sensors.
(3) A non-transitory computer readable storage medium storing a physiological information measurement program causes a computer to execute processing includes: obtaining, from a plurality of sensors that are connected to an air bag containing air and measure pressures received by the air bag from a subject, signals related to the measured pressures; and determining at least one of deterioration and failure of at least one of the sensors based on the signals related to the plurality of pressures obtained from the plurality of sensors.

Signals related to measured pressures are obtained from a plurality of sensors connected to an air bag containing air, and at least one of deterioration and failure of at least one sensor is determined based on the obtained signals related to the plurality of pressures. This makes it possible to determine whether the change in the measured pressure waveform is caused by the deterioration or failure of the sensor or by a change in condition of a measurement subject.

### BRIEF DESCRIPTION OF DRAWINGS

FIG. 1 is a diagram illustrating a schematic configuration of a physiological information measurement system.

FIG. 2 is a block diagram illustrating a schematic configuration of a physiological information measurement device.

FIG. 3 is a block diagram illustrating a schematic configuration of an analysis unit.

FIG. 4 is a schematic diagram illustrating a display screen of an operation display unit.

FIG. 5 is a flowchart illustrating an operation of the physiological information measurement device.

FIG. 6 is a subroutine flowchart of step S103 in the flowchart of FIG. 5.

FIG. 7 is a diagram illustrating a pressure waveform output from each sensor.

FIG. 8 is a diagram illustrating a static pressure waveform.

FIG. 9 is a diagram illustrating a dynamic pressure waveform.

FIG. 10 is a schematic diagram illustrating data display on the operation display unit.

FIG. 11 is a block diagram illustrating a schematic configuration of a second modification of the physiological information measurement device.

FIG. 12 is a block diagram illustrating a schematic configuration of a third modification of the physiological information measurement device.

### DESCRIPTION OF EMBODIMENTS

Hereinafter, a physiological information measurement system, a physiological information measurement method, and a physiological information measurement program according to the presently disclosed subject matter will be described in detail with reference to the drawings. In the drawings, the same elements are denoted by the same reference numerals, and the redundant description thereof is omitted.

### Configuration of Physiological Information Measurement System

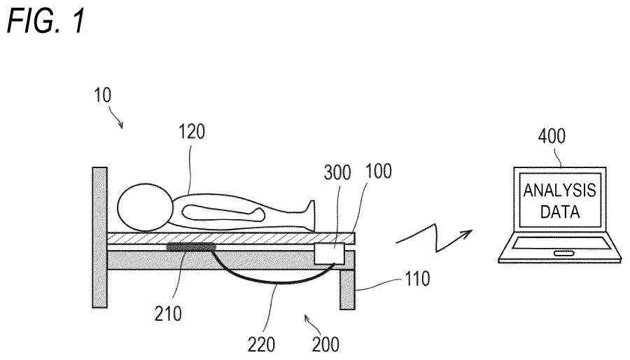

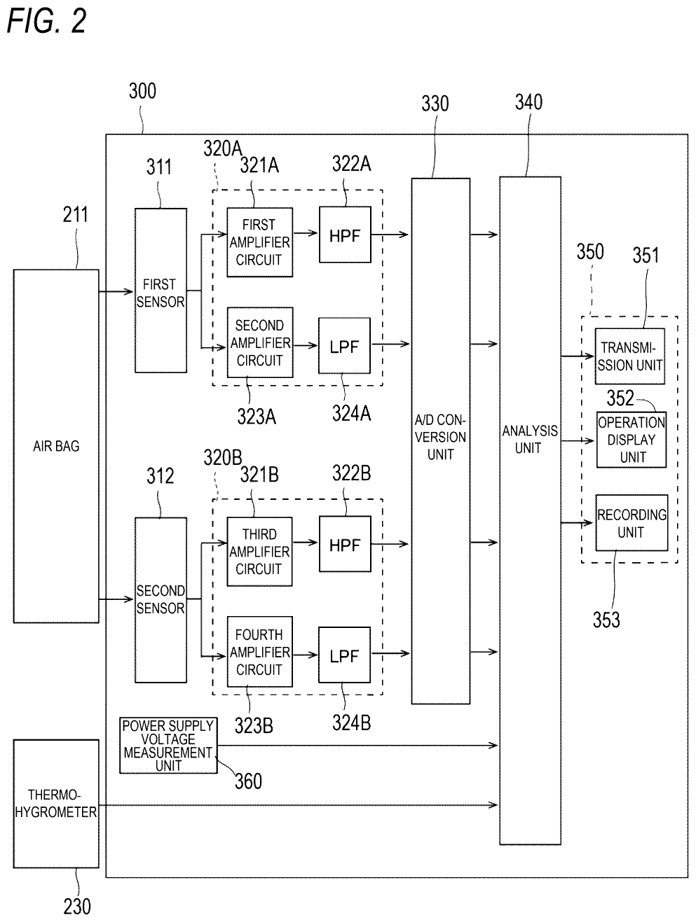

FIG. 1 is a diagram illustrating a schematic configuration of a physiological information measurement system 10 according to an embodiment. FIG. 2 is a block diagram illustrating a schematic configuration of a physiological information measurement device 300. In the present embodiment, for example, it is assumed that in a medical facility such as a hospital, a nursing home, or the like, respiration and/or a heart beat of a patient or a user (hereinafter, also referred to as a “subject 120”) are measured in a state in which the subject 120 is lying on a bed 110 on which a mattress 100 is laid. The physiological information measurement system 10 is a system mainly intended to noninvasively measure a respiration rate and the heart rate of the subject 120 who is lying on the bed 110, and continuously display and record the measured respiration rate and heart rate, and may be used, for example, in a general hospital ward of a hospital or a general home house.

The physiological information measurement system 10 can include a mat 210, a tube 220, and the physiological information measurement device 300. The physiological information measurement system 10 can further include a thermo-hygrometer 230. The thermo-hygrometer 230 constitutes a temperature and humidity measurement unit. The physiological information measurement system 10 may include only the physiological information measurement device 300. The physiological information measurement system 10 may include the mat 210, the tube 220, the physiological information measurement device 300, the thermo-hygrometer 230, and a terminal device 400. The thermo-hygrometer 230 may be incorporated in the physiological information measurement device 300 of the physiological information measurement system 10.

The mat 210 can include a sealed air bag 211. The air bag 211 has a discharge port for discharging air contained therein, and one end of a tube 220 is connected to the discharge port. The air in the air bag 211 is discharged to the physiological information measurement device 300 through the tube 220. The other end of the tube 220 is connected to the physiological information measurement device 300. A length of the tube 220 is not limited, and the tube 220 may not come out of the physiological information measurement device 300. The air bag 211 is formed of a material (for example, resin, rubber, or the like) that has airtightness and is elastically deformable by a pressure of the contained air. The air bag 211 can include an elastic body including a compressible porous body (for example, sponge) therein, and air is taken in from outside by a restoring force of the elastic body to fill the air bag.

The mat 210 can be laid, for example, at a position corresponding to the back (chest and abdomen) of the subject 120 who is lying on the bed 110 between a lower surface of the mattress 100 and an upper surface of a bed board of the bed 110 in a state where the air bag 211 is sufficiently filled with air. When the subject 120 lies on the bed 110 (gets in bed), a load due to the subject 120 on the bed 110 and the mattress 100 and a pressure due to a body motion of the subject 120 including a chest motion accompanying a respiration motion and minute vibration accompanying a heart beat motion are applied to the mat 210. Accordingly, a volume of the air bag 211 changes in accordance with the pressure received from the subject 120, so that a pressure change is transmitted to the physiological information measurement device 300 through the tube 220. The physiological information measurement device 300 may be installed on the bed 110.

The mat 210 may be laid in a position corresponding to the back (chest and abdomen) of the subject 120 on the mattress 100 laid on the bed 110. In this case, the load due to the subject 120 lying on the bed 110 and the pressure due to the body motion of the subject 120 are applied to the mat 210.

The thermo-hygrometer 230 measures a temperature (an air temperature) and humidity, and transmits measurement results to the physiological information measurement device 300. The thermo-hygrometer 230 is preferably installed at a position closer to the mat 210. The measurement result of the thermo-hygrometer 230 is used for checking an environmental state around the patient and checking an influence on a measurement performance of a first sensor 311 and a second sensor 312 to be described later.

### Configuration of Physiological Information Measurement Device 300

As illustrated in FIG. 2, the physiological information measurement device 300 can includes the first sensor 311, the second sensor 312, a first signal processing unit 320A, a second signal processing unit 320B, an A/D conversion unit 330, an analysis unit 340, a power supply voltage measurement unit 360, and a system unit 350. The first signal processing unit 320A, the second signal processing unit 320B, and the A/D conversion unit 330 constitute an obtaining unit. The analysis unit 340 constitutes a determination unit and an abnormality occurrence time measurement unit.

### Configuration of First Sensor 311

The first sensor 311 is connected to the other end of the tube 220, converts the pressure change of the air from the air bag 211 into an electrical signal, and outputs the electrical signal as a signal (a pressure waveform) related to the pressure to the signal processing unit 320. The first sensor 311 is, for example, a sensor using a piezoelectric element or a pressure sensor such as a semi-conductor sensor. A pressure sensor capable of measuring a negative pressure is used as the first sensor 311, and a body motion change can be measured even when a negative pressure is generated.

### Configuration of Second Sensor 312

The second sensor 312 is connected to the other end of the tube 220, converts the pressure change of the air from the air bag 211 into an electrical signal, and outputs the electrical signal as a signal (a pressure waveform) related to the pressure to the signal processing unit 320. The second sensor 312 may be a sensor of the same type (for example, the same product) as the first sensor 311. Hereinafter, when simply referred to as a “sensor”, the first sensor 311 and the second sensor 312 are not distinguished from each other, and either or both of the sensors are meant.

### Configuration of First Signal Processing Unit 320A

The first signal processing unit 320A processes the measurement result of the first sensor 311 to obtain a signal related to a dynamic pressure and a static pressure of the subject 120. The dynamic pressure is a body motion component including a respiration motion and a heart beat motion of the subject 120. The static pressure is a pressure caused by a change in the load of the subject 120. The first signal processing unit 320A includes a first amplifier circuit 321A, a high-pass filter (HPF) 322A, a second amplifier circuit 323A, and a low-pass filter (LPF) 324A.

The first amplifier circuit 321A amplifies the electrical signal from the first sensor 311 to a predetermined voltage level and outputs the amplified signal. The HPF 322A extracts an alternating-current component having a predetermined first cutoff frequency fc1 or higher from the electrical signal amplified by the first amplifier circuit 321A, and outputs the alternating-current component to the A/D conversion unit 330. The first cutoff frequency fc1 is a frequency at which a frequency component corresponding to the dynamic pressure can pass while a frequency component corresponding to the static pressure being sufficiently cut off. For example, fc1 may be set to be a frequency at which a frequency component derived from respiration can pass. The frequency derived from respiration may be set to be, for example, about 0.1 [Hz] to 0.4 [Hz] for an adult, based on information on the subject 120.

The second amplifier circuit 323A amplifies the electrical signal from the first sensor 311 to a predetermined voltage level and outputs the amplified signal. The LPF 324A extracts an alternating-current (and a direct-current) component having a predetermined second cutoff frequency fc2 or lower from the electrical signal amplified by the second amplifier circuit 323A, and outputs the alternating-current (and the direct-current) component to the A/D conversion unit 330. The second cutoff frequency fc2 is a frequency at which a frequency component corresponding to the static pressure can pass while a frequency component corresponding to the dynamic pressure being sufficiently cut off. The first and second cutoff frequencies may be the same value or different values (fc1>fc2).

### Configuration of Second Signal Processing Unit 320B

Same as or similarly to the first signal processing unit 320A, the second signal processing unit 320B processes the measurement result of the second sensor 312 to obtain a signal related to the dynamic pressure and the static pressure of the subject 120. The second signal processing unit 320B can include a third amplifier circuit 321B, an HPF 322B, a fourth amplifier circuit 323B, and an LPF 324B.

The third amplifier circuit 321B amplifies the electrical signal from the second sensor 312 to a predetermined voltage level and outputs the amplified signal. The HPF 322B extracts an alternating-current component having a predetermined third cutoff frequency fc3 or higher from the electrical signal amplified by the third amplifier circuit 321B, and outputs the alternating-current component to the A/D conversion unit 330. The third cutoff frequency fc3 is a frequency at which a frequency component corresponding to the dynamic pressure can pass while a frequency component corresponding to the static pressure being sufficiently cut off. In the present embodiment, the third cutoff frequency fc3 may have the same value as the first cutoff frequency fc1.

The fourth amplifier circuit 323B amplifies the electrical signal from the second sensor 312 to a predetermined voltage level and outputs the amplified signal. The LPF 324B extracts an alternating-current (and a direct-current) component having a predetermined fourth cutoff frequency fc4 or lower from the electrical signal amplified by the fourth amplifier circuit 323B, and outputs the alternating-current (and the direct-current) component to the A/D conversion unit 330. The fourth cutoff frequency fc4 is a frequency at which a frequency component corresponding to the static pressure can pass while a frequency component corresponding to the dynamic pressure being sufficiently cut off. In the present embodiment, the fourth cutoff frequency fc4 may have the same value as the second cutoff frequency fc2.

The first cutoff frequency fc1 and the third cutoff frequency fc3 may be the same as or different from the second cutoff frequency fc2 and the fourth cutoff frequency fc4.

### Configuration of A/D Conversion Unit 330

The A/D conversion unit 330 converts the electrical signal (an analog signal) of the dynamic pressure input from the HPF 322A into a digital signal of the dynamic pressure, and outputs the digital signal as a first dynamic pressure waveform to the analysis unit 340. The A/D conversion unit 330 converts the electrical signal (an analog signal) of the static pressure input from the LPF 324A into a digital signal of the static pressure, and outputs the digital signal as a first static pressure waveform to the analysis unit 340. The A/D conversion unit 330 converts the electrical signal (an analog signal) of the dynamic pressure input from the HPF 322B into a digital signal of the dynamic pressure, and outputs the digital signal as a second dynamic pressure waveform to the analysis unit 340. The A/D conversion unit 330 converts the electrical signal (an analog signal) of the static pressure input from the LPF 324B into a digital signal of the static pressure, and outputs the digital signal as a second static pressure waveform to the analysis unit 340.

Each of an output signal of the first amplifier circuit 321A (or the second amplifier circuit 323A) and an output signal of the third amplifier circuit 321B (or the fourth amplifier circuit 323B) may be further input to the A/D conversion unit 330, converted into a pressure signal of a digital signal, and output the pressure signal to the analysis unit 340 as a pressure waveform (hereinafter also referred to as an “original pressure waveform”) before filtering by the HPF 322 or the LPF 324. As will be described later, the original pressure waveform may be used in step S201 of FIG. 6 to determine whether the signal of any of the sensors has a value outside a design range.

### Configuration of Analysis Unit 340

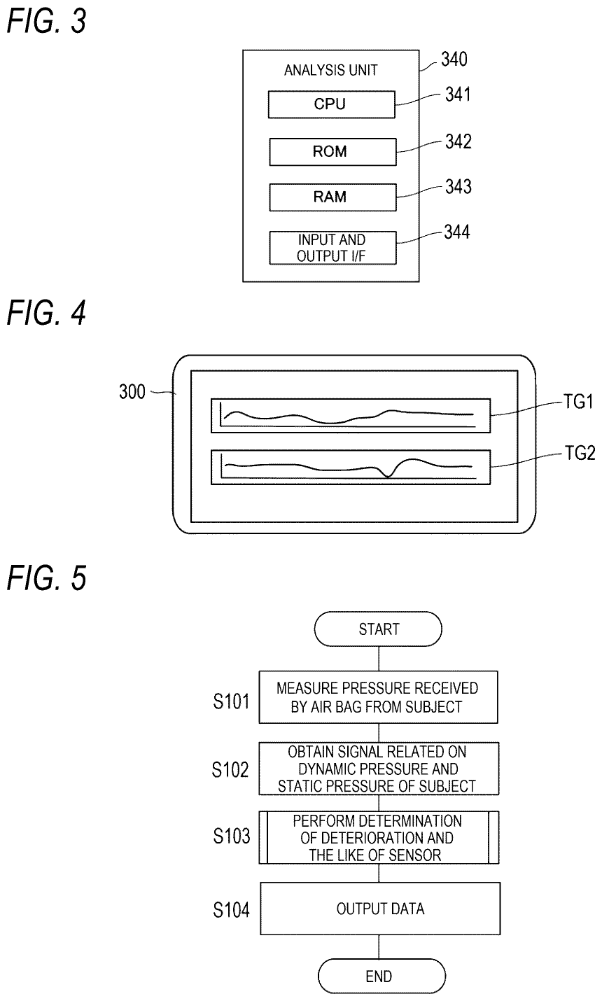

FIG. 3 is a block diagram illustrating a schematic configuration of the analysis unit 340. The analysis unit 340 can include a central processing unit (CPU) 341, a read only memory (ROM) 342, a random access memory (RAM) 343, and an input and output I/F 344. The CPU 341, the ROM 342, and the RAM 343 constitute a computer. The analysis unit 340 may include at least one processor and at least one memory.

The CPU 341 implements various functions by loading a physiological information measurement program stored in advance in the ROM 342 into the RAM 343 and executing the program.

The ROM 342 is a non-volatile memory. The ROM 342 stores programs such as an operating system (OS) and a physiological information measurement program, and various parameters necessary for calculation processing of the CPU 341. For example, a solid state drive (SSD) or a hard disk drive (HDD) that stores a calculation result by the CPU 341 and various types of data may be further included.

The RAM 343 is a volatile memory and temporarily stores various types of data.

The input and output I/F 344 is an input and output interface that transmits and receives data to and from the system unit 350.

The analysis unit 340 calculates the respiration waveform, the respiration rate, a heartbeat waveform, and the heart rate of the subject 120 based on the first dynamic pressure waveform output from the A/D conversion unit 330. More specifically, the analysis unit 340 generates the respiration waveform based on a waveform obtained by extracting a frequency component derived from respiration from the first dynamic pressure waveform, and calculates the respiration rate of the subject 120 by a predetermined analysis algorithm for calculating the respiration rate. For the extraction of the frequency component derived from the respiration, for example, in a case where the subject 120 is an adult, a digital filter or the like that allows passage of a waveform component of about 0.1 [Hz] to 1.0 [Hz] can be used. In addition, the analysis unit 340 generates the heartbeat waveform based on the waveform obtained by extracting the frequency component derived from the heart beat from the first dynamic pressure waveform, and calculates the heart rate of the subject 120 by a predetermined analysis algorithm for calculating the heart rate.

Based on the first dynamic pressure waveform and/or the first static pressure waveform, the analysis unit 340 determines a state (for example, an in bed position, a body position (decubitus position), turning over, out of bed and in bed, or the like) of the subject 120. For example, the analysis unit 340 determines in bed and out of bed of the subject 120 based on the first static pressure waveform. When the first static pressure waveform is greater than a predetermined determination threshold value, the analysis unit 340 determines that the subject 120 is in bed, and when the first static pressure waveform is less than the determination threshold value, the analysis unit 340 determines that the subject 120 is out of bed. The determination threshold value may be stored in the ROM 342 in advance. In addition, the analysis unit 340 can more accurately detect the state of the subject 120 by analyzing the first dynamic pressure waveform and the first static pressure waveform in combination. For example, the analysis unit 340 may be configured to determine that the subject 120 is in bed when the respiration and/or the heart beat of the subject 120 can be confirmed based on the first dynamic pressure waveform in addition to the first static pressure waveform being greater than the predetermined determination threshold value.

The analysis unit 340 may be configured to calculate the determination threshold value for determining the state of the subject 120 based on the first dynamic pressure waveform and/or the first static pressure waveform of the subject 120. For example, the analysis unit 340 may be configured to calculate a determination threshold value for determining in bed and out of bed of the subject 120 on the bed based on the first dynamic pressure waveform and the first static pressure waveform of the subject 120.

The analysis unit 340 calculates each determination threshold value based on the first dynamic pressure waveform and the first static pressure waveform of the subject 120 obtained at the start of pressure measurement by the first sensor 311 or at predetermined time intervals. For example, at a timing when the subject 120 gets in bed and respiration measurement is started, the analysis unit 340 calculates a determination threshold value when determining in bed and out of bed of bed of the subject 120 based on the first dynamic pressure waveform and the first static pressure waveform of the subject 120. Accordingly, an appropriate determination threshold value according to weight of the subject 120 can be applied to state determination. Alternatively, the analysis unit 340 calculates a determination threshold value when determining in bed and out of bed of the subject 120 based on the first dynamic pressure waveform and the first static pressure waveform obtained in a certain period from the timing of every predetermined time. Accordingly, even when the user of the bed 110 changes from the subject 120 to another subject, the determination threshold value according to the other subject 120 can be applied.

The analysis unit 340 estimates the weight of the subject 120 based on the change in the first static pressure waveform. The analysis unit 340 estimates the weight of the subject 120 based on a look-up table or a relational expression representing a relationship between the magnitude of the value of the first static pressure waveform and the weight of the subject 120. For example, a user who is a medical worker measures the weight and the magnitude of the value of the static pressure waveform corresponding to the weight for each of a plurality of subjects 120 in advance, and stores the measured weight and the magnitude of the value of the static pressure waveform in the ROM 342 as a look-up table or a relational expression.

The analysis unit 340 determines whether a weight equal to or greater than a load capacity is applied to the mat 210 based on the first static pressure waveform. The value of the static pressure waveform corresponding to the load capacity is stored in the ROM 342 in advance.

The analysis unit 340 determines whether an estimated value of the weight of the subject 120 or a tendency of the respiration waveform and the heartbeat waveform rapidly changes. For example, when the estimated value of the weight of the subject 120 rapidly changes, the subject 120 may have been out of bed or replaced with another person having a different weight. In a case where the tendency of the respiration waveform and the heartbeat waveform rapidly changes, conditions of the subject 120 may have been changed or the person has been replaced with another person having a different tendency of the respiration waveform and the heartbeat waveform.

As described above, the physiological information on the subject 120 is measured based on the first dynamic pressure waveform and the first static pressure waveform. As will be described later, the second dynamic pressure waveform and the second static pressure waveform may be used to determine at least one of deterioration and failure of at least one sensor of the first sensor 311 and the second sensor 312, and may be used to the physiological information measurement of the subject.

The analysis unit 340 determines at least one of deterioration and failure of at least one sensor based on signals related to a plurality of pressures. Specifically, for example, the analysis unit 340 determines deterioration and failure of at least one of the first sensor 311 and the second sensor 312 based on the first dynamic pressure waveform and the second dynamic pressure waveform. The analysis unit 340 may determine deterioration and failure of at least one of the first sensor 311 and the second sensor 312 based on the first static pressure waveform and the second static pressure waveform. The analysis unit 340 may determine deterioration and failure of at least one of the first sensor 311 and the second sensor 312 based on the first dynamic pressure waveform, the second dynamic pressure waveform, the first static pressure waveform, and the second static pressure waveform. The analysis unit 340 may determine at least one of deterioration and failure of at least one sensor of the three or more sensors based on signals related to three or more pressures of the three or more sensors. In addition, the signals related to three or more pressures of three or more sensors may be used for physiological information measurement of a subject. Hereinafter, in order to simplify the description, a case where the analysis unit 340 determines at least one of deterioration and failure of at least one sensor of the two sensors, that is, the two sensors of the first sensor 311 and the second sensor 312, based on the signals related to the plurality of pressures will be described as an example. Hereinafter, determining at least one of deterioration and failure of the sensor is also simply referred to as “determination of deterioration and the like”. The determination that at least one sensor has failed is also simply referred to as “failure determination”. The determination that at least one sensor has deteriorated is also simply referred to as “deterioration determination”. A specific method for the determination of deterioration and the like will be described in detail later.

### Configuration of System Unit 350

FIG. 4 is a schematic diagram illustrating a display screen of an operation display unit 352 illustrated in FIG. 2. The system unit 350 can include a transmission unit 351, the operation display unit 352, and a recording unit 353. The transmission unit 351 and the operation display unit 352 constitute a notification unit.

The transmission unit 351 transmits, to the terminal device 400, data relating to an analysis result of the respiration and the heart beat (the respiration rate and the heart rate, the respiration waveform and the heartbeat waveform, a trend graph, or the like) by the analysis unit 340, a state of the subject 120, an alarm, and the determination of deterioration and the like. The state of the subject 120 includes, for example, in bed and out of bed, the body position (decubitus position), the trend graph of a change in the body position, a position, the weight estimated value, and a health state of the subject 120. The alarm includes an alarm of occurrence of a body motion, occurrence of deterioration of the sensor, and occurrence of a failure of the sensor. The data related to the determination of deterioration and the like includes, for example, a signal related to the pressure, the first dynamic pressure waveform, the first static pressure waveform, the second dynamic pressure waveform, the second static pressure waveform, the temperature (air temperature) and humidity measured by the thermo-hygrometer 230, and data of the power supply voltage measured by the power supply voltage measurement unit 360.

The transmission unit 351 transmits an alarm of the occurrence of the deterioration of the sensor and the occurrence of the failure of the sensor, thereby notifying at least one of the deterioration of the sensor and the failure of the sensor. As will be described later, deterioration of a static pressure function and a dynamic pressure function of the sensor and failure of the static pressure function and the dynamic pressure function of the sensor can be determined. In this case, the transmission unit 351 transmits an alarm of occurrence of the deterioration of the static pressure function and the dynamic pressure function of the sensor and occurrence of the failure of the static pressure function and the dynamic pressure function of the sensor. Thus, the deterioration and failure of the static pressure function and the dynamic pressure function of the sensor are notified.

The user can check the data transmitted from the transmission unit 351 in the terminal device 400 and store the data in a storage device. The terminal device 400 may be, for example, a personal computer, a smartphone, or a tablet terminal.

The transmission unit 351 and the terminal device 400 are communicably connected to each other via a wireless/wired network. Examples of the network can include a local area network (LAN), a wide area network (WAN), and a USB. As a communication standard of the network, for example, Ethernet (registered trademark), Wi-Fi (registered trademark), Bluetooth (registered trademark), specific low-power radio for medical telemeter, or 5G may be used.

The operation display unit 352 can include, for example, a touch panel and various keys and switches, and receives instructions from the user, various settings, information on the patient (subject 120), and the like. The instruction by the user includes, for example, an instruction to determine deterioration or the like. The various settings include, for example, settings related to a data output method and a display method. The information on the patient includes sex, age, past medical history, and the like of the patient.

The operation display unit 352 includes a display and a speaker (not illustrated) disposed on one surface of a case of the physiological information measurement device 300, and outputs data related to the analysis result of the respiration and the heart beat by the analysis unit 340, the state of the subject 120, the alarm, and determination of deterioration and the like. The output of the operation display unit 352 can include, for example, display of data on the display, audio output to the speaker, and print output to a printer. The operation display unit 352 may be connected to an external printer. FIG. 4 illustrates a case where a trend graph TG1 of the respiration rate and a trend graph TG2 of the heart rate are displayed on the display. Details of data display on the display will be described later.

The recording unit 353 can include, for example, a large-capacity storage device such as an SSD, and records various types of data including the above data.

The power supply voltage measurement unit 360 measures and outputs power supply voltages of the first sensor 311 and the second sensor 312. A known voltmeter (including a voltage measurement circuit) may be used as the power supply voltage measurement unit 360. The power supply voltage measurement unit 360 may measure and output power supply voltages of the first signal processing unit 320A and the second signal processing unit 320B.

### Physiological Information Measurement Method

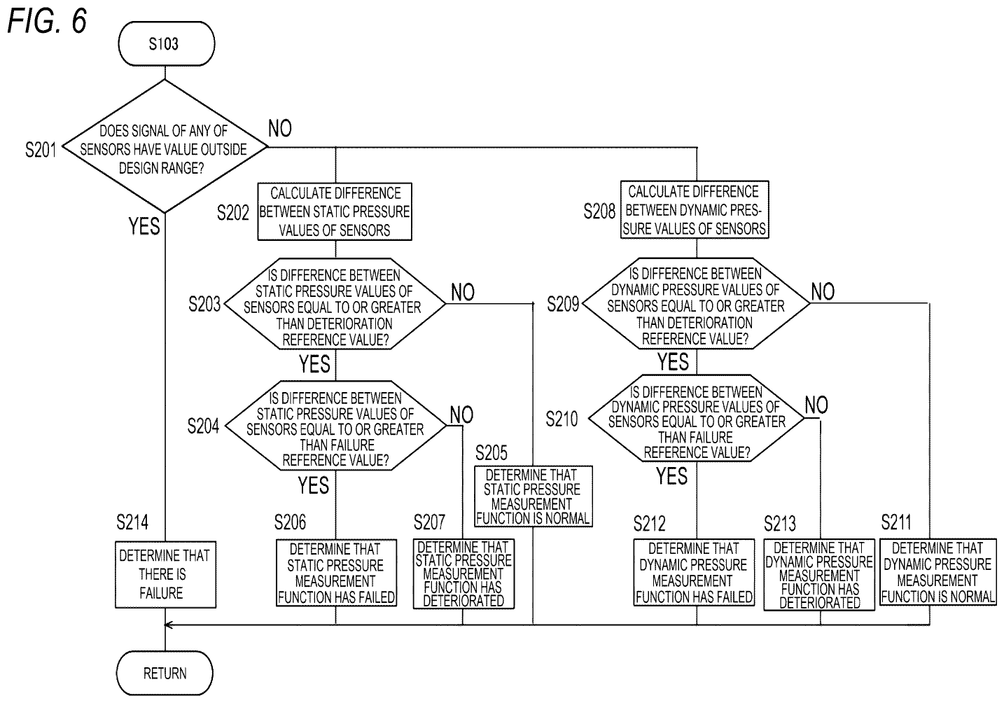

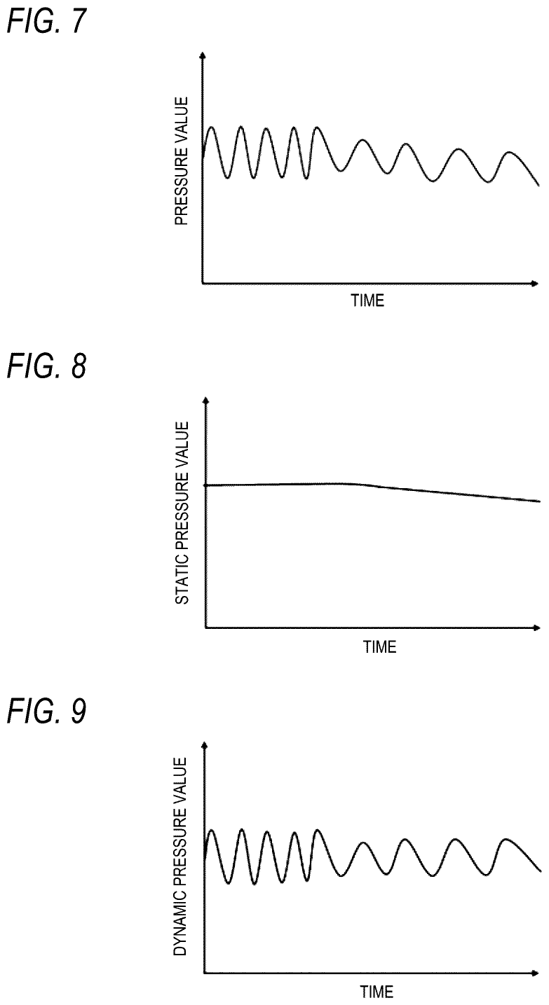

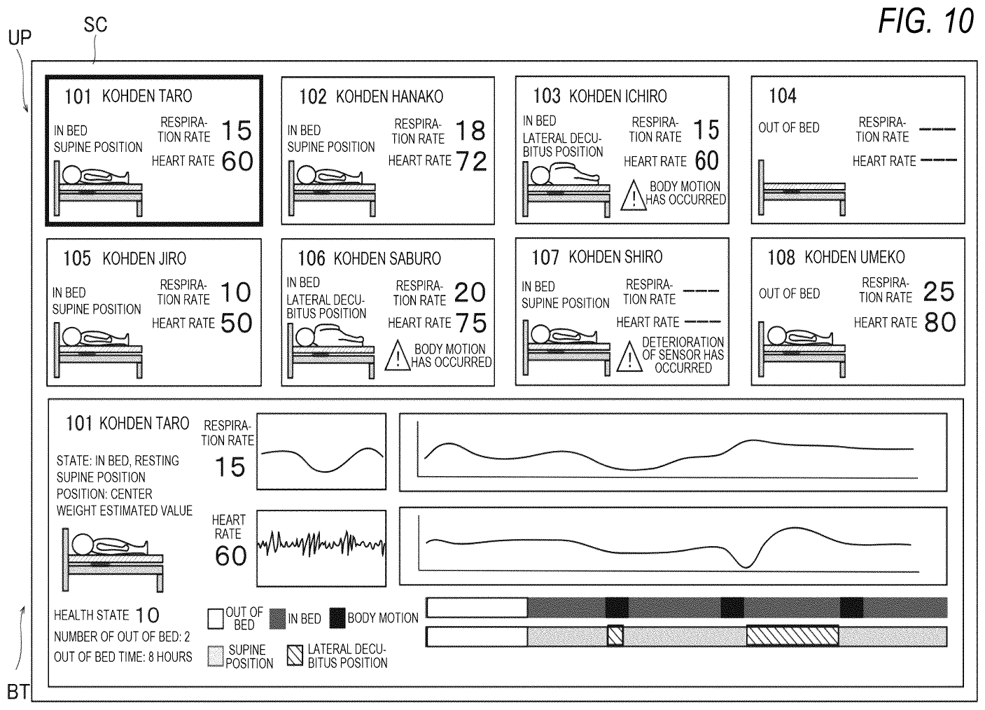

FIG. 5 is a flowchart illustrating an operation of the physiological information measurement device 300 according to the present embodiment. FIG. 6 is a subroutine flowchart of step S103 in the flowchart of FIG. 5. This flowchart is realized by the CPU 341 executing the physiological information measurement program. FIG. 7 is a diagram illustrating a pressure waveform output from each sensor. FIG. 8 is a diagram illustrating the static pressure waveform. FIG. 9 is a diagram illustrating the dynamic pressure waveform. FIG. 10 is a schematic diagram illustrating data display on the operation display unit 352.

### Preparation of Measurement

First, prior to the measurement of the physiological information on the subject 120, the user arranges the mat 210 at a position corresponding to the back (chest and abdomen) of the subject 120 between the lower surface of the mattress 100 and the upper surface of the bed board of the bed 110 or on the mattress 100. After the subject 120 gets in the bed 110, the user instructs the physiological information measurement device 300 to start measurement (for example, presses a measurement start button). Alternatively, the measurement is started when the power supply of the physiological information measurement device 300 is turned on.

### Measurement of Physiological Information

As illustrated in FIG. 5, when the measurement is started, the first sensor 311 and the second sensor 312 measure the pressure from the air bag 211 as a pressure waveform, which is generated due to the body motion of the subject 120 (S101).

Next, the first signal processing unit 320A, the second signal processing unit 320B, and the A/D conversion unit 330 obtain digital signals of the dynamic pressure waveform and the static pressure waveform as signals related to the dynamic pressure and the static pressure of the subject 120 (S102). Specifically, the first signal processing unit 320A, the second signal processing unit 320B, and the A/D conversion unit 330 obtain the first dynamic pressure waveform, the first static pressure waveform, the second dynamic pressure waveform, and the second static pressure waveform.

Next, the analysis unit 340 performs determination relating to deterioration of the sensor or the like (S103).

### Details of Processing for Determination of Deterioration And the Like of Sensor (S103)

As illustrated in FIG. 6, the analysis unit 340 determines whether the signal of at least one of the sensors has a value outside the design range (S201). The design range constitutes a predetermined threshold value range. The value outside the design range is, for example, a value exceeding a lower limit value or an upper limit value of the value of the pressure waveform in design. Whether the signal of the sensor is a value outside the design range can be determined by, for example, whether the value of the pressure waveform output from the sensor exceeds the lower limit value or the upper limit value (that is, a predetermined threshold value range) of the design range. Specifically, the analysis unit 340 determines whether a pressure waveform is obtained from the first sensor 311 or the second sensor 312, and whether a pressure waveform having an abnormal value outside the design range is obtained. More specifically, the analysis unit 340 determines, for example, whether any one of the first dynamic pressure waveform, the first static pressure waveform, the second dynamic pressure waveform, and the second static pressure waveform is not obtained, and whether any one of the first dynamic pressure waveform, the first static pressure waveform, the second dynamic pressure waveform, and the second static pressure waveform has an abnormal value outside the design range. When the output signal of the first amplifier circuit 321A (or the second amplifier circuit 323A) and the output signal of the third amplifier circuit 321B (or the fourth amplifier circuit 323B) are input to the A/D conversion unit 330, converted into original pressure signals of digital signals, and output to the analysis unit 340, it may be determined whether any one of the two original pressure signals is obtained, and whether a pressure waveform having an abnormal value outside the design range is obtained. The predetermined threshold value range may be a value obtained by adding an adjustment to the design range.

When it is determined that the signal of at least one of the sensors has a value outside the design range (S201: YES), the analysis unit 340 determines that there is a failure (S214).

When it is determined that the signal of any sensor does not have a value outside the design range (the pressure waveforms within the design range are obtained from all the sensors) (S201: NO), the analysis unit 340 calculates a difference between the values between the static pressure waveforms of the sensors (hereinafter, also referred to as a “difference between static pressure values”) (S202).

The analysis unit 340 determines whether the difference between the static pressure values of the respective sensors is equal to or greater than a deterioration reference value of a static pressure measurement function (S203). The deterioration reference value of the static pressure measurement function constitutes a first threshold value. Specifically, the analysis unit 340 determines whether the difference between the values of the first static pressure waveform and the second static pressure waveform is equal to or greater than the deterioration reference value of the static pressure measurement function. More specifically, for example, the analysis unit 340 determines whether an absolute value of the difference between the values of the first static pressure waveform and the second static pressure waveform at the same time (the difference between the static pressure values) is equal to or greater than the deterioration reference value of the static pressure measurement function. The difference between the static pressure values includes not only the absolute value of the difference at the same time but also an absolute value of the difference between average values in the same time zone, for example. That is, in step S203, the analysis unit 340 may determine whether the absolute value of the difference between the average value of the first static pressure waveform and the average value of the second static pressure waveform in the same time zone is equal to or greater than the deterioration reference value of the static pressure measurement function. When three or more sensors are used, the analysis unit 340 may determine whether a mutual difference between a plurality of static pressure waveforms based on a plurality of pressure waveforms measured by the plurality of sensors (a difference between static pressure values) is equal to or greater than the deterioration reference value of the static pressure measurement function. The deterioration reference value of the static pressure measurement function may be set to a value that is more appropriate for experiments from a viewpoint of the accuracy of the determination of deterioration and the like. The deterioration reference value of the static pressure measurement function can be set in further consideration of the type, specification, manufacturing variation, and the like of the sensor.

When it is determined that the difference between the static pressure values of the sensors is equal to or greater than the deterioration reference value of the static pressure measurement function (S203: YES), the analysis unit 340 determines whether the difference between the static pressure values of the sensors is equal to or greater than a failure reference value of the static pressure measurement function (S204). The failure reference value of the static pressure measurement function constitutes a second threshold value. Specifically, the analysis unit 340 determines whether the difference between the first static pressure waveform and the second static pressure waveform (the difference between the static pressure values) is equal to or greater than the failure reference value of the static pressure measurement function. More specifically, for example, the analysis unit 340 determines whether the absolute value of the difference between the first static pressure waveform and the second static pressure waveform at the same time (the difference between the static pressure values) is equal to or greater than the failure reference value of the static pressure measurement function. When three or more sensors are used, the analysis unit 340 may determine whether the mutual difference between the plurality of static pressure waveforms based on the plurality of pressure waveforms measured by the plurality of sensors (the difference between the static pressure values) is equal to or greater than the failure reference value of the static pressure measurement function. The failure reference value of the static pressure measurement function may be set to a value that is more appropriate for experiments from the viewpoint of the accuracy of the determination of deterioration and the like. The failure reference value of the static pressure measurement function can be set in further consideration of the type, specification, manufacturing variation, and the like of the sensor.

When it is determined that the difference between the static pressure values of the sensors is equal to or greater than the failure reference value of the static pressure measurement function (S204: YES), the analysis unit 340 determines that the static pressure measurement function has failed (S206). In this case, the analysis unit 340 may simply determine that the sensor has failed.

When it is determined that the difference between the static pressure values of the sensors is less than the failure reference value of the static pressure measurement function (S204: NO), the analysis unit 340 determines that the static pressure measurement function has deteriorated (S207). In this case, the analysis unit 340 may simply determine the sensor has deteriorated.

When it is determined in step S203 that the difference between the static pressure values of the sensors is less than the deterioration reference value of the static pressure measurement function (S203: NO), the control unit 340 determines that the static pressure measurement function is normal (S205).

From step S202 to step S207, when the difference between the first static pressure waveform and the second static pressure waveform is equal to or greater than the deterioration reference value (the first threshold value) of the static pressure measurement function and less than the failure reference value (the second threshold value) of the static pressure measurement function, the analysis unit 340 determines that the static pressure measurement function of the sensor has deteriorated. When the difference between the first static pressure waveform and the second static pressure waveform is equal to or greater than the failure reference value (the second threshold value) of the static pressure measurement function, the analysis unit 340 determines that the static pressure measurement function of the sensor has failed. The failure reference value of the static pressure measurement function is naturally greater than the deterioration reference value of the static pressure measurement function.

When a state in which the difference between the first static pressure waveform and the second static pressure waveform is equal to or greater than the deterioration reference value (the first threshold value) of the static pressure measurement function and less than the failure reference value (the second threshold value) of the static pressure measurement function continues for a predetermined first time, the analysis unit 340 may determine that the difference between the first static pressure waveform and the second static pressure waveform is equal to or greater than the deterioration reference value (the first threshold value) of the static pressure measurement function and less than the failure reference value (the second threshold value) of the static pressure measurement function. The predetermined first time may be set to a value that is more appropriate for experiments from the viewpoint of the accuracy of the determination of deterioration and the like. The predetermined first time may be set in further consideration of the type, specification, and the like of the sensor. The predetermined first time may be set to, for example, about 30 minutes (for example, a time in a range of 20 minutes to 40 minutes).

When a state in which the difference between the first static pressure waveform and the second static pressure waveform exceeds the failure reference value (second threshold value) of the static pressure measurement function continues for a predetermined second time, the analysis unit 340 may determine that the difference between the first static pressure waveform and the second static pressure waveform exceeds the failure reference value (second threshold value) of the static pressure measurement function. The predetermined second time may be set to a value that is more appropriate for experiments from the viewpoint of the accuracy of the determination of deterioration and the like. The predetermined second time can be set in further consideration of the type, specification, and the like of the sensor. The predetermined second time may be set to, for example, about 30 minutes (for example, a time in a range of 20 minutes to 40 minutes).

Step S202 to step S207 and step S208 to step S213 can be performed in parallel.

The analysis unit 340 calculates a difference between the values of the dynamic pressure waveforms of the sensors (hereinafter, also referred to as a “difference between dynamic pressure values”) (S208).

The analysis unit 340 determines whether the difference between the dynamic pressure values of the respective sensors is equal to or greater than a deterioration reference value of a dynamic pressure measurement function (S209). The deterioration reference value of the dynamic pressure measurement function constitutes a third threshold value. Specifically, the analysis unit 340 determines whether the difference between the values of the first dynamic pressure waveform and the second dynamic pressure waveform is equal to or greater than the deterioration reference value of the dynamic pressure measurement function. More specifically, for example, the analysis unit 340 determines whether an absolute value of the difference between the values of the first dynamic pressure waveform and the second dynamic pressure waveform at the same time (the difference between the dynamic pressure values) is equal to or greater than the deterioration reference value of the dynamic pressure measurement function. The difference between the dynamic pressure values includes not only the absolute value of the difference at the same time but also an absolute value of the difference between average values in the same time zone, for example. That is, in step S209, the analysis unit 340 may determine whether the absolute value of the difference between the average value of the first dynamic pressure waveform and the average value of the second dynamic pressure waveform in the same time zone is equal to or greater than the deterioration reference value of the dynamic pressure measurement function. When three or more sensors are used, the analysis unit 340 may determine whether a mutual difference between a plurality of dynamic pressure waveforms based on a plurality of pressure waveforms measured by the plurality of sensors (a difference between dynamic pressure values) is equal to or greater than the deterioration reference value of the dynamic pressure measurement function. The deterioration reference value of the dynamic pressure measurement function may be set to a value that is more appropriate for experiments from the viewpoint of the accuracy of the determination of deterioration and the like. The deterioration reference value of the dynamic pressure measurement function can be set in further consideration of the type, specification, manufacturing variation, and the like of the sensor.

When it is determined that the difference between the dynamic pressure values of the sensors is equal to or greater than the deterioration reference value of the dynamic pressure measurement function (S209: YES), the analysis unit 340 determines whether a difference between the dynamic pressure values of the sensors is equal to or greater than a failure reference value of a dynamic pressure measurement function (S210). The failure reference value of the dynamic pressure measurement function constitutes a fourth threshold value. Specifically, the analysis unit 340 determines whether the difference between the first dynamic pressure waveform and the second dynamic pressure waveform (the difference between the dynamic pressure values) is equal to or greater than the failure reference value of the dynamic pressure measurement function. More specifically, for example, the analysis unit 340 determines whether the absolute value of the difference between the first dynamic pressure waveform and the second dynamic pressure waveform at the same time (the difference between the dynamic pressure values) is equal to or greater than the failure reference value of the dynamic pressure measurement function. When three or more sensors are used, the analysis unit 340 may determine whether the mutual difference between a plurality of dynamic pressure waveforms based on a plurality of pressure waveforms measured by the plurality of sensors (the difference between the dynamic pressure values) is equal to or greater than the failure reference value of the dynamic pressure measurement function. The failure reference value of the dynamic pressure measurement function may be set to a value that is more appropriate for experiments from the viewpoint of the accuracy of the determination of deterioration and the like. The failure reference value of the dynamic pressure measurement function can be set in further consideration of the type, specification, manufacturing variation, and the like of the sensor.

When it is determined that the difference between the dynamic pressure values of the sensors is equal to or greater than the failure reference value of the dynamic pressure measurement function (S210: YES), the analysis unit 340 determines that the dynamic pressure measurement function has failed (S212). In this case, the analysis unit 340 may simply determine that the sensor has failed.

When it is determined that the difference between the dynamic pressure values of the sensors is less than the failure reference value of the dynamic pressure measurement function (S210: NO), the analysis unit 340 determines that the dynamic pressure measurement function has deteriorated (S213). In this case, the analysis unit 340 may simply determine the sensor has deteriorated.

When it is determined in step S209 that the difference between the dynamic pressure values of the sensors is less than the deterioration reference value of the dynamic pressure measurement function (S209: NO), the control unit 340 determines that the dynamic pressure measurement function is normal (S211).

Through step S208 to step S213, when the difference between the first dynamic pressure waveform and the second dynamic pressure waveform is equal to or greater than the deterioration reference value (the third threshold value) of the dynamic pressure measurement function and less than the failure reference value (the fourth threshold value) of the dynamic pressure measurement function, the analysis unit 340 determines that the dynamic pressure measurement function of the sensor has deteriorated. When the difference between the first dynamic pressure waveform and the second dynamic pressure waveform is equal to or greater than the failure reference value (the fourth threshold value) of the dynamic pressure measurement function, the analysis unit 340 determines that the dynamic pressure measurement function of the sensor has failed. The failure reference value of the dynamic pressure measurement function is naturally greater than the deterioration reference value of the dynamic pressure measurement function.

When a state in which the difference between the first dynamic pressure waveform and the second dynamic pressure waveform is equal to or greater than the deterioration reference value (the third threshold value) of the dynamic pressure measurement function and less than the failure reference value (the fourth threshold value) of the dynamic pressure measurement function continues for a predetermined third time, the analysis unit 340 may determine that the difference between the first dynamic pressure waveform and the second dynamic pressure waveform is equal to or greater than the third threshold value and less than the fourth threshold value. The predetermined third time may be set to a value that is more appropriate for experiments from the viewpoint of the accuracy of the determination of deterioration and the like. The predetermined third time may be set in further consideration of the type, specification, and the like of the sensor. The predetermined third time may be set to, for example, about 30 minutes (for example, a time in a range of 20 minutes to 40 minutes). When a state in which the difference between the first dynamic pressure waveform and the second dynamic pressure waveform exceeds the fourth threshold value continues for a predetermined fourth time, the analysis unit 340 may determine that the difference between the first dynamic pressure waveform and the second dynamic pressure waveform exceeds the fourth threshold value. The predetermined fourth time may be set to a value that is more appropriate for experiments from the viewpoint of the accuracy of the determination of deterioration and the like. The predetermined fourth time may be set in further consideration of the type, specification, and the like of the sensor. The predetermined fourth time may be set to, for example, about 30 minutes (for example, a time in a range of 20 minutes to 40 minutes).

The analysis unit 340 measures, as an abnormality occurrence time, a time from a time point at which it is determined that there is a failure or deterioration by the determination of deterioration and the like, and records the abnormality occurrence time in the recording unit 353.

After the power supply of the physiological information measurement device 300 is turned on, the analysis unit 340 may determinate the deterioration and the like continuously, intermittently, or when an instruction for determination is received. When the deterioration and the like is intermittently determined, for example, the deterioration and the like may be determined at any time interval in a range of one minute to several hours. The analysis unit 340 can receive the instruction for the determination by, for example, selecting a button displayed on the display of the operation display unit 352. The analysis unit 340 may receive the instruction for determination by receiving an instruction for determination transmitted from the terminal device 400 to the physiological information measurement device 300 when a button displayed on the display of the terminal device 400 is selected.

The analysis unit 340 determines whether there is a failure in the power supply based on the power supply voltage measured by the power supply voltage measurement unit 360, and may determine the deterioration and the like when it is determined that there is no failure in the power supply. That is, when it is determined that there is a failure in the power supply, the analysis unit 340 may not perform the determination of deterioration and the like. When the power supply voltage is not within a predetermined threshold value range, the analysis unit 340 may determine that the power supply has failed.

The analysis unit 340 may perform the determination of deterioration and the like based on the temperature and the humidity measured by the thermo-hygrometer 230. Specifically, for example, the analysis unit 340 corrects the first dynamic pressure waveform, the first static pressure waveform, the second dynamic pressure waveform, and the second static pressure waveform based on the temperature and the humidity, and performs the determination of deterioration and the like based on the corrected first dynamic pressure waveform, first static pressure waveform, second dynamic pressure waveform, and second static pressure waveform.

### Data Display

Returning again to the flowchart illustrated in FIG. 5, the operation display unit 352 outputs data (step S104). For example, the operation display unit 352 outputs, to the display, data related to an analysis result of the respiration and the heart beat (the respiration rate and the heart rate, the waveform, the trend graph, or the like) by the analysis unit 340, the state of the subject 120, the alarm, and the determination of deterioration and the like. In the present embodiment, when the deterioration of the sensor occurs, the sensor outputs an alarm of the deterioration of the sensor regarding the subject 120 installed on the bed 110. When the failure of the sensor occurs, the sensor outputs an alarm of the failure of the sensor regarding the subject 120 installed on the bed 110. It is assumed that the respiration and/or the heart beat of the subject 120 is measured while the subject 120 is lying on the bed. The physiological information measurement system 10 is a system mainly intended to noninvasively measure the respiration rate and the heart rate of the subject 120 who is lying on the bed 110 and continuously display and record the measured respiration rate and heart rate, and may be used, for example, in a general hospital ward of a hospital.

As illustrated in FIG. 10, for example, the operation display unit 352 displays the respiration rate, the heart rate, in bed and out of bed, and the body position (decubitus position) of the patient (the subject 120), and the alarm, together with the hospital room number of the patient for each patient, on the display of the terminal device 400 or in an upper stage portion UP of a screen SC of the display. In an example illustrated in FIG. 10, regarding Mr. Kohden Ichiro in Room No. 103 and Mr. Kohden Saburo in Room No. 106, an alarm indicating the occurrence of a body motion is displayed, and regarding Mr. Kohden Shiro in Room No. 107, an alarm indicating the occurrence of deterioration of a sensor is displayed. For example, the operation display unit 352 may display characters of “body motion has occurred” together with a warning mark as an alarm of the occurrence of a body motion, for example, when the subject 120 is moving violently and the waveform is disturbed. For example, the operation display unit 352 may display characters of “deterioration of sensor has occurred” together with a warning mark as an alarm of occurrence of deterioration of the sensor.

In the present embodiment, the analysis unit 340 is configured to determine in bed and out of bed of the subject 120, based on a change in the static pressure signal and/or the dynamic pressure signal, and the operation display unit 352 accurately displays the respiration rate and the heart rate only when the subject 120 is in bed. This prevents erroneous calculation and display of the respiration rate and the heart rate when a bed user is out of bed.

When the analysis unit 340 determines that a weight equal to or greater than the load capacity is applied to the mat 210, the operation display unit 352 notifies the user of the determination. Accordingly, it is possible to prevent the measurement of the respiration and the heart beat from continuing in a state in which the weight equal to or greater than the load capacity is applied to the mat 210 and accuracy cannot be guaranteed.

When it is determined that the estimated value of the weight of the subject 120 or the tendency of the respiration waveform and the heartbeat waveform rapidly changes, the operation display unit 352 notifies the user of this determination. When the estimated value of the weight of the subject 120 or the tendency of the respiration waveform and the heartbeat waveform rapidly changes, for example, there is a possibility that the condition of the subject 120 has changed or the subject 120 has been replaced with another person, and therefore the operation display unit 352 may notify the user that there is a possibility that the condition of the subject 120 changes or the user of the bed 110 is replaced.

The operation display unit 352 displays, for example, for Mr. Kohden Taro in Room No. 101, in a lower stage portion BT of the screen SC, in addition to the information of the upper stage portion UP, detailed information regarding the information on in bed and out of bed, the position and the weight estimated value (specific numerical values are omitted in the figure) of Mr. Kohden Taro on the bed, the graph related to the respiration and the heart beat, the information on the health state, the information on the decubitus position, and the like. From the data displayed on the screen SC, the user can immediately grasp that Mr. Kohden Taro is in a resting state and is in bed in a supine position at a center of the bed, the respiration rate is 15, the heart rate is 60, the health state is 10, the number of times of getting out of the bed is 2, and out of bed time is 8 hours. Instead of the number of out of bed times and out of bed time, the number of in bed times and in bed time may be displayed. The information on health states is, for example, an index (score) evaluated on a scale of 1 to 10 relative to the health states of the subject 120, and is calculated based on changes in the respiration rate and the heart rate, and the respiration waveform and the heartbeat waveform.

The graph related to the respiration and the heart beat may be, for example, real-time waveforms of the respiration and the heart beat or the trend graphs of the respiration rate and heart rate. In the example in FIG. 10, for example, histories of in bed, out of bed, and the body motion in one day are displayed in band graph shapes as the information on in bed and out of bed, and a history of the body position (decubitus position) (supine position or lateral decubitus position), in one day is displayed in a band graph shape as the information on the body position (decubitus position).

### First Modification

The first sensor 311 and the second sensor 312 may be different types of sensors. For example, one of the first sensor 311 and the second sensor 312 is a sensor using a piezo element, and the other is a semi-conductor sensor.

In the present modification, in order to calculate the difference between the waveforms, Adjustment is made in advance to match the maximum value of the output of the first sensor 311 and the maximum value of the output of the second sensor 312.

The sensors of the same type generally have close deterioration rates. On the other hand, different types of sensors may have relatively different deterioration rates. Therefore, by determining the deterioration of the sensors based on the differences among the plurality of waveforms obtained from the different types of sensors, it is possible to prevent a decrease in the determination accuracy of the deterioration of the sensor due to the fact that the deterioration rates of the sensors of the same type are close to each other, and to improve the determination accuracy of the deterioration of the sensor.

### Second Modification

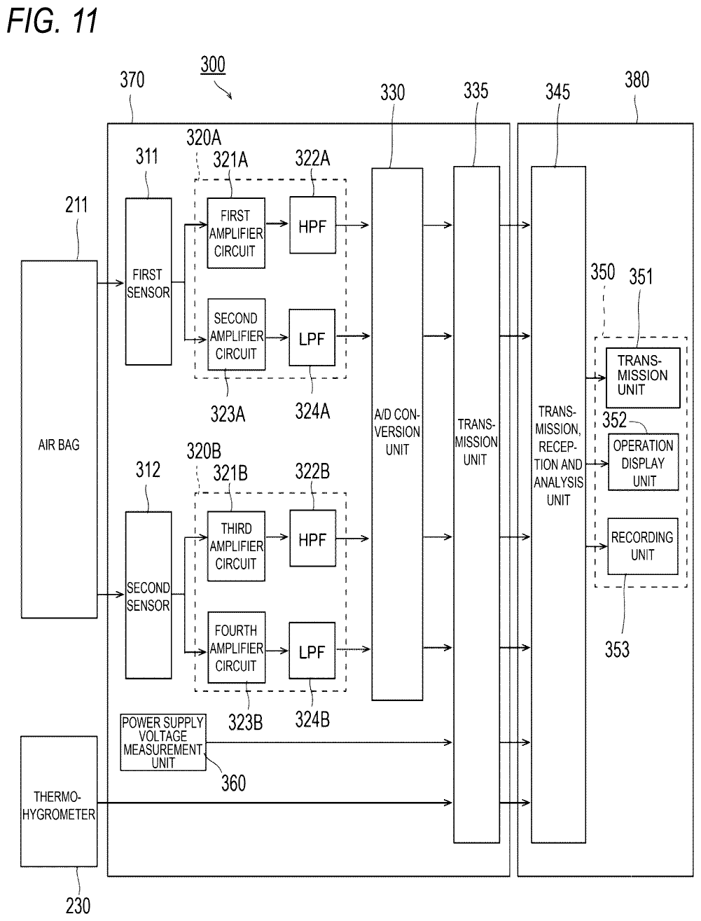

FIG. 11 is a block diagram illustrating a schematic configuration of a second modification of the physiological information measurement device 300 illustrated in FIG. 1.

In the present modification, the physiological information measurement device 300 can include two parts: a first body unit 370 including the first sensor 311, the second sensor 312, the first signal processing unit 320A, the second signal processing unit 320B, the A/D conversion unit 330, and a transmission unit 335; and a second body unit 380 including a transmission, reception and analysis unit 345 and the system unit 350. The first body unit 370 and the second body unit 380 may be disposed in different places in a hospital, for example, and may communicate with each other. For example, the first body unit 370 is disposed on the bed 110 in a hospital room of a patient, and the second body unit 380 may be carried by a user. The second body unit 380 may be a computer such as a server.

The configurations of the first sensor 311, the second sensor 312, the first signal processing unit 320A, the second signal processing unit 320B, and the A/D conversion unit 330 are the same as the corresponding configurations of the embodiment described above, and detailed description thereof will be omitted. The transmission unit 335 transmits, to the second body unit 380, the first dynamic pressure waveform, the first static pressure waveform, the second dynamic pressure waveform, and the second static pressure waveform converted by the A/D conversion unit 330, the power supply voltage output from the power supply voltage measurement unit 360, and the temperature and humidity received from the thermo-hygrometer 230. The transmission, reception and analysis unit 345 of the second body unit 380 receives the first dynamic pressure waveform, the first static pressure waveform, the second dynamic pressure waveform, the second static pressure waveform, the power supply voltage, the temperature, and the humidity from the transmission unit 335.

The transmission, reception and analysis unit 345 executes the functions executed by the analysis unit 340 of the embodiment described above.

### Third Modification

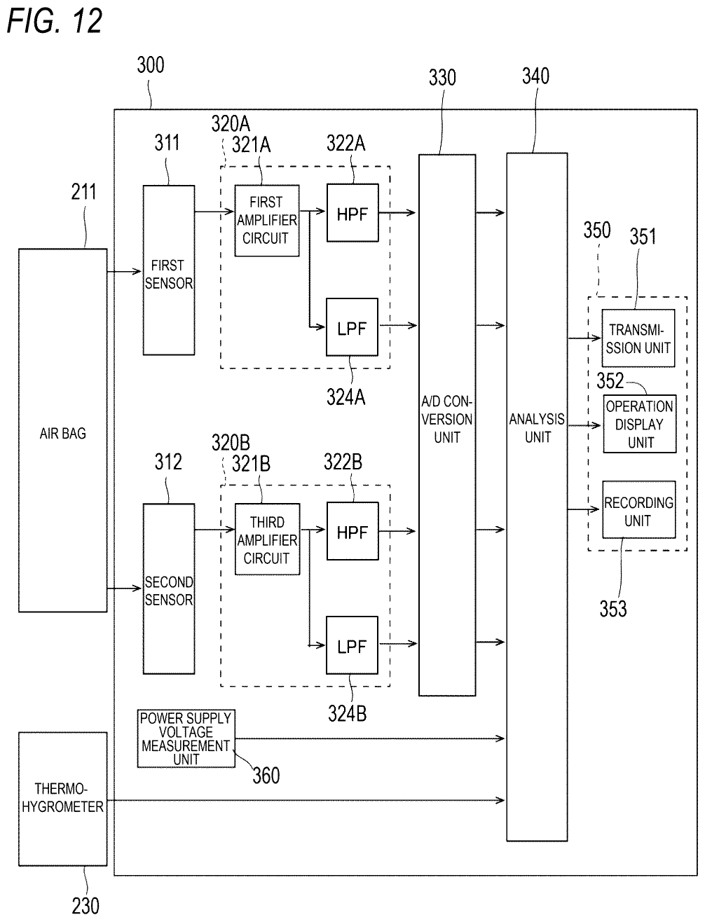

FIG. 12 is a block diagram illustrating a schematic configuration of a third modification of the physiological information measurement device 300 illustrated in FIG. 1.

In this modification, the second amplifier circuit and the fourth amplifier circuit are omitted. The LPF 324A extracts the alternating-current (and the direct-current) component having the predetermined second cutoff frequency fc2 or lower from the electrical signal amplified by the first amplifier circuit 321A, and outputs the alternating-current (and the direct-current) component to the A/D conversion unit 330. The LPF 324B extracts the alternating-current (and the direct-current) component having the predetermined fourth cutoff frequency fc4 or lower from the electrical signal amplified by the third amplifier circuit 321B, and outputs the alternating-current (and the direct-current) component to the A/D conversion unit 330.

The physiological information measurement device 300 according to the present modification may be implemented by the first body unit 370 and the second body unit 380 as in the second modification.

The physiological information measurement device 300 has the following effects.

Signals related to measured pressures are obtained from a plurality of sensors connected to an air bag containing air, and at least one of deterioration and failure of at least one sensor is determined based on the obtained signals related to the plurality of pressures. This makes it possible to determine whether the change in the measured pressure waveform is caused by the deterioration or failure of the sensor or by a change in condition of a measurement subject.

In addition, the signal related to the pressure is a pressure waveform, and at least one of deterioration and failure of the sensor is determined based on the differences among the plurality of pressure waveforms. The deterioration and failure of the sensor can be determined easily and with high accuracy.

The plurality of sensors are two different types of sensors. Accordingly, by using the same type of sensor, it is possible to prevent a decrease in the determination accuracy of deterioration of the sensor due to the close deterioration rates of the plurality of sensors.

When the pressure waveforms within the predetermined threshold value range are obtained from all of the plurality of sensors and the difference between the plurality of static pressure waveforms based on the plurality of obtained pressure waveforms is equal to or greater than the first threshold value and less than the second threshold value, it is determined that the sensor has deteriorated. When pressure waveforms within the predetermined threshold value range are not obtained from at least one of the plurality of sensors or the pressure waveforms within the predetermined threshold value range are obtained from all of the plurality of sensors, and when the mutual difference between the plurality of static pressure waveforms based on the plurality of obtained pressure waveforms is equal to or greater than the second threshold value, it is determined that the sensor has failed. Accordingly, the deterioration and the failure of the sensor can be determined more easily and with high accuracy.

When the pressure waveforms within the predetermined threshold value range are obtained from all of the plurality of sensors and the mutual difference between the plurality of dynamic pressure waveforms based on the plurality of obtained pressure waveforms is equal to or greater than the third threshold value and less than the fourth threshold value, it is determined that the sensor has deteriorated. When the pressure waveforms within the predetermined threshold value range are not obtained from at least one of the plurality of sensors or the pressure waveforms within the predetermined threshold value range are obtained from all of the plurality of sensors, and when the mutual difference between the plurality of dynamic pressure waveforms based on the plurality of obtained pressure waveforms is equal to or greater than the fourth threshold value, it is determined that the sensor has failed. Accordingly, the deterioration and the failure of the sensor can be determined more easily and with high accuracy.

When the pressure waveforms within the predetermined threshold value range are obtained from all of the plurality of sensors and the mutual difference between the plurality of static pressure waveforms based on the obtained plurality of pressure waveforms is equal to or greater than the first threshold value and less than the second threshold value, it is determined that the static pressure measurement function of the sensor has deteriorated. When the pressure waveforms within the predetermined threshold value range are not obtained from at least one of the plurality of sensors or the pressure waveforms within the predetermined threshold value range are obtained from all of the plurality of sensors, and when the mutual difference between the plurality of static pressure waveforms based on the plurality of obtained pressure waveforms is equal to or greater than the second threshold value, it is determined that the static pressure measurement function of the sensor has failed. Accordingly, the deterioration and failure of the static pressure measurement function of the sensor can be determined more easily and with high accuracy.

When the pressure waveforms within the predetermined threshold value range are obtained from all of the plurality of sensors and the mutual difference between the plurality of dynamic pressure waveforms based on the plurality of obtained pressure waveforms is equal to or greater than the third threshold value and less than the fourth threshold value, it is determined that the dynamic pressure measurement function of the sensor has deteriorated. When the pressure waveforms within the predetermined threshold value range are not obtained from at least one of the plurality of sensors or the pressure waveforms within the predetermined threshold value range are obtained from all of the plurality of sensors, and when the mutual difference between the plurality of dynamic pressure waveforms based on the plurality of obtained pressure waveforms is equal to or greater than the fourth threshold value, it is determined that the dynamic pressure measurement function of the sensor has failed. Accordingly, the deterioration and failure of the static pressure measurement function of the sensor can be determined more easily and with high accuracy. In addition, the time from the time point at which it is determined that the sensor has deteriorated or failed is measured and recorded. Thereby, for example, the information for more effectively improving the determination accuracy of the deterioration and the failure of the sensor can be easily obtained.

At least one of deterioration and failure of the sensor is determined continuously, intermittently, or when an instruction for determination is received. Accordingly, the deterioration and the failure of the sensor can be determined flexibly and efficiently.

Whether there is a failure in the power supply is determined based on the voltage of the power supply measured by the power supply voltage measurement unit, and when it is determined that there is no failure in the power supply, at least one of deterioration and failure of the sensor is determined. Accordingly, it is possible to prevent a decrease in the determination accuracy of the deterioration and the failure of the sensor due to the failure in the power supply.

At least one of deterioration and failure of the sensor is determined based on the temperature and humidity measured by the temperature and humidity measurement unit. Accordingly, it is possible to prevent a decrease in the determination accuracy of deterioration and failure of the sensor due to fluctuations in temperature and humidity.

When the state in which the mutual difference between the plurality of static pressure waveforms based on the plurality of pressure waveforms is equal to or greater than the first threshold value and less than the second threshold value continues for the predetermined first time, it is determined that the mutual difference between the plurality of static pressure waveforms is equal to or greater than the first threshold value and less than the second threshold value. When the state in which the mutual difference between the plurality of static pressure waveforms based on the plurality of pressure waveforms is equal to or greater than the second threshold value continues for the predetermined second time, it is determined that the mutual difference between the plurality of static pressure waveforms is equal to or greater than the second threshold value. Accordingly, it is possible to prevent a decrease in the determination accuracy of deterioration and failure of the sensor due to temporary instability of the obtained pressure waveform.

When the state in which the mutual difference between the plurality of dynamic pressure waveforms based on the plurality of pressure waveforms is equal to or greater than the third threshold value and less than the fourth threshold value continues for the predetermined third time, it is determined that the mutual difference between the plurality of dynamic pressure waveforms is equal to or greater than the third threshold value and less than the fourth threshold value. When the state in which the mutual difference between the plurality of dynamic pressure waveforms based on the plurality of pressure waveforms is equal to or greater than the fourth threshold value continues for the predetermined fourth time, it is determined that the mutual difference between the plurality of dynamic pressure waveforms is equal to or greater than the fourth threshold value. Accordingly, it is possible to prevent a decrease in the determination accuracy of deterioration and failure of the sensor due to temporary instability of the obtained pressure waveform.

The notification unit that notifies at least one of deterioration and failure of the determined sensor is included. This makes it possible to easily and quickly know the occurrence of deterioration and failure of the sensor.

Although the physiological information measurement device 300, the physiological information measurement method, and the physiological information measurement program according to the presently disclosed subject matter have been described above, the presently disclosed subject matter is not limited to the embodiment described above.

For example, in the flowchart of FIG. 6, step S202 and step S204 may be omitted, and when it is determined in step S201 that the signal of any sensor does not have a value outside the design range (S201: NO), step S203 and step S204 may be performed in parallel.

When the first sensor 311 and the second sensor 312 are sensors that cannot measure the static pressure (when all the sensors mounted in the physiological information measurement system 10 are sensors that cannot measure the static pressure), step S202 to step S207 may be omitted in the flowchart of FIG. 6.

A part or all of the functions implemented by the physiological information measurement program in the embodiment described above may be implemented by hardware such as an electric circuit.

A physiological information measurement method and a non-transitory computer readable storage medium storing a physiological information measurement program according to the presently disclosed subject matter include the following configurations.
(1) A physiological information measurement method including:
obtaining, from a plurality of sensors that are connected to an air bag containing air and measure pressures received by the air bag from a subject, signals related to the measured pressures; and
determining at least one of deterioration and failure of at least one of the sensors based on the signals related to the plurality of pressures obtained from the plurality of sensors.
(2) The physiological information measurement method according to (1), in which
the signal related to the pressure is a pressure waveform, and
in the determination, at least one of deterioration and failure of the sensor is determined based on a difference between values of a plurality of the pressure waveforms.
(3) The physiological information measurement method according to (1), in which
the plurality of sensors are two different types of sensors.
(4) The physiological information measurement method according to (1), in which
the signal related to the pressure is a pressure waveform, and
in the determination,
it is determined that the sensor has deteriorated when the pressure waveforms within a predetermined threshold value range are obtained from all of the plurality of sensors and a mutual difference between a plurality of static pressure waveforms based on the obtained plurality of pressure waveforms is equal to or greater than a first threshold value and less than a second threshold value, and
it is determined that the sensor has failed when the pressure waveforms within the predetermined threshold value range are not obtained from at least one of the plurality of sensors or the pressure waveforms within the predetermined threshold value range are obtained from all of the plurality of sensors, and when a mutual difference between the plurality of static pressure waveforms based on the plurality of obtained pressure waveforms is equal to or greater than the second threshold value.
(5) The physiological information measurement method according to (1), in which
the signal related to the pressure is a pressure waveform, and
in the determination,
it is determined that the sensor has deteriorated when the pressure waveforms within a predetermined threshold value range are obtained from all of the plurality of sensors and a mutual difference between a plurality of dynamic pressure waveforms based on the obtained plurality of pressure waveforms is equal to or greater than a third threshold value and less than a fourth threshold value, and
it is determined that the sensor has failed when the pressure waveforms within the predetermined threshold value range are not obtained from at least one of the plurality of sensors or the pressure waveforms within the predetermined threshold value range are obtained from all of the plurality of sensors, and when a mutual difference between the plurality of dynamic pressure waveforms based on the plurality of obtained pressure waveforms is equal to or greater than the fourth threshold value.
(6) The physiological information measurement method according to (4), in which
in the determination,
it is determined that a static pressure measurement function of the sensor has deteriorated when the pressure waveforms within the predetermined threshold value range are obtained from all of the plurality of sensors and the mutual difference between the plurality of static pressure waveforms based on the plurality of obtained pressure waveforms is equal to or greater than the first threshold value and less than the second threshold value, and
it is determined that the static pressure measurement function of the sensor has failed when the pressure waveforms within the predetermined threshold value range are not obtained from at least one of the plurality of sensors or the pressure waveforms within the predetermined threshold value range are obtained from all of the plurality of sensors, and when the mutual difference between the plurality of static pressure waveforms based on the plurality of obtained pressure waveforms is equal to or greater than the second threshold value.
(7) The physiological information measurement method according to (5), in which
in the determination,
it is determined that a dynamic pressure measurement function of the sensor has deteriorated when the pressure waveforms within the predetermined threshold value range are obtained from all of the plurality of sensors and the mutual difference between the plurality of dynamic pressure waveforms based on the plurality of obtained pressure waveforms is equal to or greater than the third threshold value and less than the fourth threshold value, and
it is determined that the dynamic pressure measurement function of the sensor has failed when the pressure waveforms within the predetermined threshold value range are not obtained from at least one of the plurality of sensors or the pressure waveforms within the predetermined threshold value range are obtained from all of the plurality of sensors, and when the mutual difference between the plurality of dynamic pressure waveforms based on the plurality of obtained pressure waveforms is equal to or greater than the fourth threshold value.
(8) The physiological information measurement method according to (1), further including:
measuring and recording a time from a time point at which it is determined that the sensor has deteriorated or failed, in the determination.
(9) The physiological information measurement method according to (1), in which
in the determination, at least one of deterioration and failure of the sensor is determined continuously, intermittently, or when an instruction for determination is received.
(10) The physiological information measurement method according to (1), further including:
measuring a voltage of a power supply, in which
in the determination, whether there is a failure in the power supply is determined based on the voltage of the power supply measured in the measuring the voltage of the power supply, and at least one of deterioration and failure of the sensor is determined when it is determined that there is no failure in the power supply.
(11) The physiological information measurement method according to (1), further including:
measuring a temperature and humidity, in which
in the determination, at least one of deterioration and failure of the sensor is determined based on the temperature and humidity measured in the measuring the temperature and the humidity.
(12) The physiological information measurement method according to (4), in which
in the determination,
it is determined that the mutual difference between the plurality of static pressure waveforms based on the plurality of obtained pressure waveforms is equal to or greater than the first threshold value and less than the second threshold value when a state in which the mutual difference between the plurality of static pressure waveforms is equal to or greater than the first threshold value and less than the second threshold value continues for a predetermined first time, and
it is determined that the mutual difference between the plurality of static pressure waveforms based on the plurality of obtained pressure waveforms is equal to or greater than the second threshold value when a state in which the mutual difference between the plurality of static pressure waveforms is equal to or greater than the second threshold value continues for a predetermined second time.
(13) The physiological information measurement method according to (5), in which
in the determination,
it is determined that the mutual difference between the plurality of dynamic pressure waveforms based on the obtained plurality of pressure waveforms is equal to or greater than the third threshold value and less than the fourth threshold value when a state in which the mutual difference between the plurality of dynamic pressure waveforms is equal to or greater than the third threshold value and less than the fourth threshold value continues for a predetermined third time, and
it is determined that the mutual difference between the plurality of dynamic pressure waveforms based on the obtained plurality of pressure waveforms is equal to or greater than the fourth threshold value when a state in which the mutual difference between the plurality of dynamic pressure waveforms is equal to or greater than the fourth threshold value continues for a predetermined fourth time.
(14) The physiological information measurement method according to (1), further including:
notifying at least one of deterioration and failure of the sensor determined in the determination.
(15) A non-transitory computer readable storage medium storing a physiological information measurement program for causing a computer to execute the physiological information measurement method according to any one of (1) to (14).

1. A physiological information measurement system comprising:
an obtaining unit configured to obtain, from a plurality of sensors that are connected to an air bag containing air and measure pressures received by the air bag from a subject, signals related to the measured pressures; and
a determination unit configured to determine at least one of deterioration and failure of at least one of the sensors based on the signals related to the pressures obtained from the plurality of sensors.

2. The physiological information measurement system according to claim 1, wherein
the signal related to the pressure is a pressure waveform, and
the determination unit determines at least one of deterioration and failure of the sensor based on a difference between values of a plurality of the pressure waveforms.

3. The physiological information measurement system according to claim 1, wherein
the plurality of sensors are provided, and
the plurality of sensors are two different types of sensors.

4. The physiological information measurement system according to claim 1, wherein
the signal related to the pressure is a pressure waveform, and
the determination unit
determines that the sensor has deteriorated when the pressure waveforms within a predetermined threshold value range are obtained from all of the plurality of sensors and a mutual difference between a plurality of static pressure waveforms based on the obtained plurality of pressure waveforms is equal to or greater than a first threshold value and less than a second threshold value, and
determines that the sensor has failed when the pressure waveforms within the predetermined threshold value range are not obtained from at least one of the plurality of sensors or the pressure waveforms within the predetermined threshold value range are obtained from all of the plurality of sensors, and when a mutual difference between the plurality of static pressure waveforms based on the plurality of obtained pressure waveforms is equal to or greater than the second threshold value.

5. The physiological information measurement system according to claim 1, wherein
the signal related to the pressure is a pressure waveform, and
the determination unit
determines that the sensor has deteriorated when the pressure waveforms within a predetermined threshold value range are obtained from all of the plurality of sensors and a mutual difference between a plurality of dynamic pressure waveforms based on the obtained plurality of pressure waveforms is equal to or greater than a third threshold value and less than a fourth threshold value, and
determines that the sensor has failed when the pressure waveforms within the predetermined threshold value range are not obtained from at least one of the plurality of sensors or the pressure waveforms within the predetermined threshold value range are obtained from all of the plurality of sensors, and when a mutual difference between the plurality of dynamic pressure waveforms based on the plurality of obtained pressure waveforms is equal to or greater than the fourth threshold value.

6. The physiological information measurement system according to claim 4, wherein
the determination unit
determines that a static pressure measurement function of the sensor has deteriorated when the pressure waveforms within the predetermined threshold value range are obtained from all of the plurality of sensors and the mutual difference between the plurality of static pressure waveforms based on the plurality of obtained pressure waveforms is equal to or greater than the first threshold value and less than the second threshold value, and
determines that the static pressure measurement function of the sensor has failed when the pressure waveforms within the predetermined threshold value range are not obtained from at least one of the plurality of sensors or the pressure waveforms within the predetermined threshold value range are obtained from all of the plurality of sensors, and when the mutual difference between the plurality of static pressure waveforms based on the plurality of obtained pressure waveforms is equal to or greater than the second threshold value.

7. The physiological information measurement system according to claim 5, wherein
the determination unit
determines that a dynamic pressure measurement function of the sensor has deteriorated when the pressure waveforms within the predetermined threshold value range are obtained from all of the plurality of sensors and the mutual difference between the plurality of dynamic pressure waveforms based on the plurality of obtained pressure waveforms is equal to or greater than the third threshold value and less than the fourth threshold value, and
determines that the dynamic pressure measurement function of the sensor has failed when the pressure waveforms within the predetermined threshold value range are not obtained from at least one of the plurality of sensors or the pressure waveforms within the predetermined threshold value range are obtained from all of the plurality of sensors, and when the mutual difference between the plurality of dynamic pressure waveforms based on the plurality of obtained pressure waveforms is equal to or greater than the fourth threshold value.

8. The physiological information measurement system according to claim 1, further comprising:
an abnormality occurrence time measurement unit configured to measure and record a time from a time point at which the determination unit determines that the sensor has deteriorated or failed.

9. The physiological information measurement system according to claim 1, wherein
the determination unit determines at least one of deterioration and failure of the sensor continuously, intermittently, or when an instruction for determination is received.

10. The physiological information measurement system according to claim 1, further comprising:
a power supply voltage measurement unit configured to measure a voltage of a power supply, wherein
the determination unit determines whether there is a failure in the power supply based on the voltage of the power supply measured by the power supply voltage measurement unit, and determines at least one of deterioration and failure of the sensor when it is determined that there is no failure in the power supply.

11. The physiological information measurement system according to claim 1, further comprising:
a temperature and humidity measurement unit configured to measure a temperature and humidity, wherein
the determination unit determines at least one of deterioration and failure of the sensor based on the temperature and humidity measured by the temperature and humidity measurement unit.

12. The physiological information measurement system according to claim 4, wherein
the determination unit
determines that the mutual difference between the plurality of static pressure waveforms based on the plurality of obtained pressure waveforms is equal to or greater than the first threshold value and less than the second threshold value when a state in which the mutual difference between the plurality of static pressure waveforms is equal to or greater than the first threshold value and less than the second threshold value continues for a predetermined first time, and
determines that the mutual difference between the plurality of static pressure waveforms based on the plurality of obtained pressure waveforms is equal to or greater than the second threshold value when a state in which the mutual difference between the plurality of static pressure waveforms is equal to or greater than the second threshold value continues for a predetermined second time.

13. The physiological information measurement system according to claim 5, wherein
the determination unit
determines that the mutual difference between the plurality of dynamic pressure waveforms based on the obtained plurality of pressure waveforms is equal to or greater than the third threshold value and less than the fourth threshold value when a state in which the mutual difference between the plurality of dynamic pressure waveforms is equal to or greater than the third threshold value and less than the fourth threshold value continues for a predetermined third time, and
determines that the mutual difference between the plurality of dynamic pressure waveforms based on the obtained plurality of pressure waveforms is equal to or greater than the fourth threshold value when a state in which the mutual difference between the plurality of dynamic pressure waveforms is equal to or greater than the fourth threshold value continues for a predetermined fourth time.

14. The physiological information measurement system according to claim 1, further comprising:
a notification unit configured to notify at least one of deterioration and failure of the sensor determined by the determination unit.

15. A physiological information measurement method comprising:
obtaining, from a plurality of sensors that are connected to an air bag containing air and measure pressures received by the air bag from a subject, signals related to the measured pressures; and
determining at least one of deterioration and failure of at least one of the sensors based on the signals related to the plurality of pressures obtained from the plurality of sensors.

16. A non-transitory computer readable storage medium storing a physiological information measurement program for causing a computer to execute processing comprising:
obtaining, from a plurality of sensors that are connected to an air bag containing air and measure pressures received by the air bag from a subject, signals related to the measured pressures; and
determining at least one of deterioration and failure of at least one of the sensors based on the signals related to the plurality of pressures obtained from the plurality of sensors.
